(19) United States
(12) Patent Application Publication
(10) Pub. No.: US 2002/0150559 A1
(43) Pub. Date: Oct. 17, 2002

(54) Induction of T cell tolerance with CD40/B7 antagonists

(76) Inventors: Mark DeBoer, Blaricum (NL); Marcel Theodorus Den Hartog, Monnickendam (NL)

Correspondence Address: ERIC P. MIRABEL, ESQ., WINSTEAD SECHREST & MINICK P.C., 2400 BANK ONE CENTER, 910 TRAVIS STREET, HOUSTON, TX, 77002, US

(21) Appl. No.: 09/978,752

(22) Filed: Oct. 15, 2001

Related U.S. Application Data
(63) Continuation of application No. 09/235,073, filed on Jan. 21, 1999, now abandoned.
Application No. 09/235,073 is a continuation of application No. PCT/NL97/00438, filed on Jul. 23, 1997.
(60) Provisional application No. 60/022,070, filed on Jul. 23, 1996.

(51) Int. Cl. A61K 48/00; C12N 5/08; C07K 16/28

(52) U.S. Cl. 424/93.6; 424/93.21; 435/372; 530/388.22

(57) ABSTRACT

The invention provides a ligand capable of binding to the human CD40 antigen and to the human CD86 antigen, and optionally to the CD80 antigen, said antigens being located on the surface of human lymphocytes, as well as vectors capable of producing the ligand and uses of the ligand in inducing T cell tolerance. Said ligand can be an antibody, which can be a trispecific diabody capable of binding to CD40 and to both CD80 and CD86, or a bispecific diabody capable of binding to human CD40 and to human CD86, or else a trispecific triabody capable of binding to CD40, CD80 and CD86.

### FIELD OF THE INVENTION

This invention relates to methods of treating diseases with CD40/B7 antagonists.

### BACKGROUND OF THE INVENTION

Activation of T cells

Activation of T cells is known to require multiple interactions with antigen presenting cells (“APC”). The TcR-CD3 complex has two functions in antigen-induced activation: a recognition function in which a specific antigen is recognised in the context of the appropriate MHC molecule, and a signalling function in which the recognition event is transmitted across the plasma membrane. However, to induce proliferation and maturation into effector cells, T cells need a second signal in addition to the one mediated by the TcR-CD3 complex. This costimulatory signal is normally provided by the cell surface of APC. Intercellular signalling after TcR/MHC-peptide interaction in the absence of the costimulatory signal results in T cell inactivation, which is known as T cell anergy or T cell unresponsiveness. Several accessory molecules present on the cell surface of T cells with known ligands on the APC have been implicated in providing the costimulatory signal in T cell activation.

The B7-CD29 costimulatory pathway

The best candidate costimulatory signal leading to full T cell activation, is generated by interaction of CD28 on the T cells with the B7 costimulatory molecules on APC. In vitro studies have demonstrated that signalling via the CD28 costimulatory pathway can prevent the induction of anergy.

To date, two members of the B7 family have been molecularly cloned and functionally characterized: B7.1 (CD80), originally named B7/BB1 and a second B7 molecule, named B70 or B7.2 (CD86). CD80 is a monomeric transmembrane glycoprotein with an apparent molecular mass of 45-65 kDa and is, like CD28, a member of the immunoglobulin superfamily (Freeman et al., J. Immunol. 143: 2714 (1989)). Initially it was reported that the expression of the CD80 molecule was restricted to activated B cells and monocytes stimulated with IFN-γ(Freedman et al., Cell Immunol. 137: 429 (1991)). More recently, CD80 expression has also been found on cultured peripheral blood dendritic cells (Young et al., J. Clin. Invest. 90:229 (1992)) and on in vitro activated T cells (Azuma et al., J. Ax Med. 177:845 (1993)). CD86 is a transmembrane glycoprotein with an apparent molecular mass of approximately 70 KDa and is also a member of the immunoglobulin superfamily (Freeman et al., Science 262:909 (1993)); Azuma et al., Nature 366:76 (1993)). The CD86 molecule seems to have a very similar distribution pattern to CD80, with the exception that induction of cell-surface expression seems to be faster and that it is present on freshly isolated monocytes.

It is clear from the literature that blocking CD80 exclusively only results in partial inhibition of T cell activation. Activation of T cells by alloantigen-expressing monocytes is predominantly dependent on CD86 costimulation. During an MLC with monocytes as stimulator cells, anti-CD86 Mab alone could strongly but not completely inhibit the proliferative response. However, only CTLA4-Ig (a soluble fusion protein consisting of the extra-cellular domain of human CTLA4 linked to human IgG [CH2 and CH3 domains], that can bind to both CD80 and CD86) or a combination of anti-CD80 plus anti-CD86 Mabs gave maximal inhibition.

CD40-CD40L costimulatory pathway:

From the above reviewed literature data it appears that other costimulatory molecules can be of crucial importance for the activation of T cells when costimulation via CD80/CD86-CD28 is blocked. One such an alternative costimulatory pathway is regulated by the interaction between CD40 on the APC and CD40L on the T cells. The CD40 molecule belongs to the TNF receptor family of type I transmembrane proteins. The members of this gene family, which include: the two receptors for TNF; the low-affinity nerve growth factor receptor; the T cell activation antigen CD27; CD30 and CD95, are characterized by sequence homology in their cysteine-rich extracellular domains (Armitage et al., Current Opinion in Immunology 6:407 (1994)). Interestingly, the known ligands for the members of the TNF receptor family are very homologous as well, forming another gene family named the TNF/CD40L gene family. Although TNF-α is a soluble cytokine, it is initially synthesized as a membrane associated molecule. Most of the members of the TNF/CD40L receptor family are type II transmembrane proteins.

CD40 is best known for its function in B cell activation. The molecule is constitutively expressed on all B cells. CD40L-CD40 interaction can stimulate the proliferation of purified B cells and, in combination with cytokines, mediate immunoglobulin production. Recent studies indicate that the distribution of the CD40 molecule is not so restricted as was originally postulated. Freshly isolated human monocytes express low levels of the CD40 molecule, which can be up-regulated by culturing in the presence of IFN-γ (Alderson et al., J. Ep. Med. 178:669 (1993)). Stimulation of monocytes via CD40 results in the secretion of pro-inflammatory cytokines such as IL-1 and TNF-α, toxic free radical intermediates such as nitric oxide, and up-regulation of the B7 costimulatory molecules. Human dendritic cells (DC) isolated from peripheral blood can also express the CD40 molecule (Caux et al., J. Exp. Med. 180:263 (1994)). Ligation of CD40 on DC results in enhanced survival of these cells when cultured in vitro. In addition, like with monocytes, stimulation of DC results in secretion of pro-inflammatory cytokines such as IL-1 and TNF-α and up-regulation of the CD80/86 co-stimulatory molecules.

All the above described observations clearly indicate that the CD40L molecule on activated T cells is an important effector molecule that mediates stimulatory effects via ligation of CD40 expressed on a variety of cell types involved in the immunoinflammatory response. However, there is also experimental evidence that the CD40L molecule can receive signals that result in the costimulation of the T cell itself. Using mouse P815 cells that can present anti-CD3 monoclonal antibody to human T cells via binding to Fc-receptors on its cell surface, it was demonstrated that CD40 transfected P815 cells could substantially induce proliferation and CTL activity of small resting human T cells (Cayabyab et al., J. Immunol. 152:1523 (1994)). This demonstrates that the CD40L-CD40 interactions is clearly bidirectional.

Prolonged transplant survival after blocking CD80/CD86-CD28:

Several recent in vivo models have shown that induction of prolonged graft acceptance is possible by interruption of the CD80/CD86-CD28 pathway. Treatment with CTLA-4 immediately after xenogeneic human pancreatic islet transplantation in mice resulted in long-term graft survival (Lenschow et al., Science 257:789 (1992)). However, 90% of fully mismatched rat cardiac allografts were rejected in rats treated intraperitoneally (IP) with CTLA4-Ig during 7 days (Turka et al., Proc. Natl. Acad. Sci. USA 89:11102 (1992)). CTLA4-Ig given intravenously (IV) at time of transplantation and then IP every other day on days 2 through 12, prolonged cardiac allograft survival in mice, but failed to prolong the survival of primary skin grafts (Pearson et al., Transplantation 57: 1701 (1994)). Blockage of the CD28-pathway with CTLA4-Ig resulted in significant prolongation of small bowel transplant survival in rats compared to controls, although all grafts were rejected after 15 days (Pescovitz et al., Transplant Proc. 26:1618 (1994)). Finally, treatment with CTLA4-Ig could reduce lethal murine GVHD in recipients of fully allogeneic bone marrow and significantly prolonged survival rates with up to 63% of mice surviving greater than 3 months post-transplantation (Blazar et al., Blood 83:3815 (1994)). The failure of CTLA4-Ig alone to induce anergy in vitro and in vivo, can most likely be explained by a persistent IL-2 production, induced by TCR triggering in combination with signalling from other accessory molecules on APC.

Prolonged transplant survival after blocking CD)40-CD40L:

Recent work has also demonstrated that blocking the CD40-CD40L pathway is strongly immunosuppressive in transplantation models. Combined treatment with allogeneic small lymphocytes or T cell-depleted small lymphocytes plus an antibody to mouse CD40L permitted indefinite pancreatic islet allograft survival in 37 out of 40 recipients that differed in major and minor histocompatibility loci (Parker et al., Proc. Nat. Acad. Sci. USA 92:9560 (1995)). From these experiments it was concluded that the effective blocking of the CD40L-CD40 interaction most likely had resulted in preventing the induction of costimulatory molecules on the small resting lymphocytes by the alloreactive host T cells. In another recent study, it was demonstrated that administration of a blocking Mab to mouse CD40L at the time of transplantation markedly prolonged survival of fully disparate murine cardiac allografts in both naive and sensitized hosts. However, when anti-CD40L therapy was delayed until postoperative day 5, anti-CD40L failed to prolong graft survival. From this study, it was concluded that anti-CD40L therapy inhibited allograft rejection primarily by interfering with T cell help for effector functions.

Long-term acceptance of allografts without signs of chronic rejection after blocking CD80/CD86-CD28 and CD 40-CD 40L:

Blocking either of the two major T cell costimulatory pathways, CD80/86-CD28 or CD40-CD40L, alone is not sufficient to permit indefinite engraftment of highly immunogenic allografts. However, it was recently shown that blocking the CD80/CD86-CD28 and CD40-CD40L pathways simultaneously effectively aborts T cell clonal expansion in vitro and in vivo, promotes long-term survival of fully allogeneic skin grafts, and inhibits the development of chronic vascular rejection of primarily vascularized cardiac allografts (Larsen et al., Nature 381:434 (1996)). In the vascularized murine cardiac allograft model, C3HJ recipients treated with CTLA4-Ig alone (Mean Survival Time (MST) 50d), anti-CD40L Mab alone (MST 70d), or the combination (MST>70d), all showed prolonged survival of BALB/c cardiac allografts compared with untreated controls (MST 12d). Interestingly, when examined histologically at day 58-62 posttransplantation, marked differences were apparent. Allografts from CTLA4-Ig-treated mice showed extensive lymphocytic infiltration, interstitial fibrosis, and severe coronary arterial intimal thickening and fibrosis, all clear signs of a chronic rejection process. Anti-CD40L-treated recipients had less lymphocytic infiltration and interstitial fibrosis, but also had coronary vasculopathy characteristic of chronic rejection. In contrast, the allografts from mice in which the CD80/86-CD28 and CD40-CD40L pathways were blocked simultaneously, the parenchyma and blood vessels were virtually indistinguishable from those found in normal BALB/c hearts.

Current concepts on immunological tolerance hold that anergy is the result of intercellular signalling after TcR/MHC-peptide interaction, in the absence of a so-called co-stimulatory signal. As described above, both the CD80/CD86-CD28 costimulatory pathway and the CD40L-CD40 costimulatory pathway are important for the activation of T cells and play a role in the prevention of anergy. Stimulation of T cells via the TcR/CD3 complex results in a low and transient expression of CD40L. It has previously been demonstrated that stimulation of T cells via TcR/CD3 and costimulation with CD80/CD86-CD28 results in a strong and prolonged expression of the CD40L molecule. Low levels of CD86 can be found constitutively on various APC populations. Furthermore, stimulation of APC via CD40 is one of the strongest signals to up-regulate CD80 and CD86. Likewise, low levels of CD40L are expressed on T cells after first encounter of antigen (TcR/CD3 activation), even without complete costimulation. This CD40L expression can not only receive a signal from the APC via CD40, but can also stimulate the APC to enhance the CD86 expression and most importantly up-regulate CD80 expression. At the same time, low levels of CD86 expression on professional APC can prevent the induction of T cell anergy and up-regulation of CD80 and CD86 expression strongly stimulates the T cells to secrete cytokines and to enhance CD40L expression. It is therefore concluded by the present inventors that these two interactions (CD40L-CD40 and CD80/CD86-CD28) are connected in the initiation and amplification of T cell mediated immune responses. Thus, only blocking the CD40L-CD40 interaction will not completely prevent activation of the T cells via CD80/CD86-CD28, and only blocking the CD80/CD86-CD28 interaction will n completely prevent the activation of the effector functions of the APC population. For optimal immunosuppression, the CD80/CD86-CD28 and CD40L-CD40 interaction need to be blocked simultaneously.

In the above described in vivo experiments a combination of a monoclonal antibody to mouse CD40L was used in combination with CTLA4-Ig to block both CD80 and CD86. However, the generation of a single pharmaceutical agent by combining the ligand binding domains of CTLA4-Ig and that of an anti-CD40L monoclonal antibody will result in the cross-linking of T cells and APC. This will undoubtedly result in the activation of the T cells, which is exactly the opposite of the desired effect. Furthermore, all of the above referenced publications clearly suggest that blocking both CD80 and CD86 results in a better immuno-suppression than blocking each of them separately. Also, it has been demonstrated that only blocking both CD80 and CD86 can result in T cell anergy. It was therefore surprising to find that in combination with an antagonistic anti-CD40 monoclonal antibody, blocking only CD86 resulted in T cell anergy despite the fact that CD80 was not blocked. This had led to the present invention that consists of pharmaceutical molecules that have the capacity to bind and block both the CD40 and the CD86 molecules.

### SUMMARY OF THE INVENTION

The invention is based on the discovery that a molecular combination of an antagonistic molecule binding to CD40 and an antagonistic molecule binding to CD86, both expressed at low levels on professional APC, can inhibit the activation of T cells and result in T cell anergy. Accordingly, this combination can be used to prevent or treat diseases or conditions in which the activation of T cells is involved, including transplant rejection, multiple sclerosis, psoriasis, rheumatoid arthritis and systemic lupus erythematosus

One embodiment of this invention is a single soluble molecule or ligand capable of binding to the human CD40 and CD86 antigens located on the surface of antigen presenting cells. A preferred embodiment is a single protein encompassing a combination of a therapeutically active antagonistic monoclonal antibody to CD40 or fragments thereof and a therapeutically active antagonistic CD86 ligand, including a monoclonal antibody to CD86, the CTLA4-Ig molecule, or fragments thereof In a related embodiment, gene therapy techniques are used to produce such a single protein in vivo.

A more preferred embodiment is a single protein encompassing a combination of a therapeutically active antagonistic monoclonal antibody to CD40 or fragments thereof and the therapeutically active antagonistic anti-CD86 monoclonal antibody Fun-1 (Nozawa et al., J. Pathol. 169:309 (1993)) or a therapeutically active fragment thereof. Again, gene therapy can be used to produce such single protein in vivo.

### DETAILED DESCRIPTION OF THE INVENTION

All publications and applications, cited previously or below are hereby incorporated by reference. One embodiment of the bispecific molecules of the invention is formed by conjugating two single chain antibodies, one derived from an antibody specific for CD40 and the other from an antibody specific for an CD86. Another embodiment is a fusion protein including a monoclonal antibody to CD40, or a fragment thereof, and an antibody to CD86, or a fragment thereof. In either case, a CTLA4-Ig molecule can be substituted for the antibody to CD86. The monoclonal antibodies used to form the bispecific molecules include, in whole or in part, as appropriate, chimeric antibodies, humanized antibodies, human antibodies, single-chain antibodies and fragments, including Fab, F(ab′)₂, Fv and other fragments which retain the antigen binding function of the parent antibody. Single chain antibodies (“ScFv”) and the method of their construction are described in U.S. Pat. No. 4,946,778.

Chimeric antibodies are produced by recombinant processes well known in the art, and have an animal variable region and a human constant region. Humanized antibodies correspond more closely to the sequence of human antibodies than do chimeric antibodies. In a humanized antibody, only the complementarity determining regions (CDRs), which are responsible for antigen binding and specificity, are non-human derived and have an amino acid sequence corresponding to the non-human antibody, and substantially all of the remaining portions of the molecule (except, in some cases, small portions of the framework regions within the variable region) are human derived and have an amino acid sequence corresponding to a human antibody. See L. Riechmann et al., Nature; 332: 323-327 1988; U.S. Pat. No. 5,225,539 (Medical Research Council); U.S. Pat. Nos. 5,585,089; 5,693,761; 5,693,762 (Protein Design Labs, Inc.).

Human antibodies can be made by several different methods, including by use of human immunoglobulin expression libraries (Stratagene Corp., La Jolla, Calif.; Cambridge Antibody Technology Ltd., London, England) to produce fragments of human antibodies (V_{H}, V_{L}, Fv, Fd, Fab, or (Fab′)₂) and use of these fragments to construct whole human antibodies by fusion of the appropriate portion thereto, using techniques similar to those for producing chimeric antibodies. Human antibodies can also be produced in transgenic mice with a human immunoglobulin genome. Such mice are available from Abgenix, Inc., Fremont, Calif., and Medarex, Inc., Annandale, N.J. In addition to connecting the heavy and light chain Fv regions to form a single chain peptide, Fab can be constructed and expressed by similar means (M. J. Evans et al., J. Immunol. Meth., 184:123-138 1995).

All of the wholly and partially human antibodies described above are less immunogenic than wholly murine or non-human-derived antibodies, as are the fragments and single chain antibodies. All these molecules (or derivatives thereof) are therefore less likely to evoke an immune or allergic response. Consequently, they are better suited for in vivo administration in humans than wholly non-human antibodies, especially when repeated or long-term administration is necessary, as may be needed for treatment with the bispecific antibodies of the invention.

U.S. Pat. No. 5,534,254 (Creative Bimolecules, Inc.) describes several different embodiments of bispecific antibodies, including linking single chain Fv with peptide couplers, including Ser-Cys, (Gly)₄-CyS, (His)₆-(Gly)₄-Cys, chelating agents, and chemical or disulfide couplings including bismaleimidohexane and bismaleimidocaproyl. Another embodiment is a dimer having single chain FvL₁ and FvH₂ linked and FvH₁ linked with FvL₂. All such linkers and couplings can be used with the bispecific antibodies of the invention.

The bispecific molecules of the invention are administered as a pharmaceutical composition at a dosage effective to inhibit the activation of T cells. The effective dosage can be readily determined in routine human clinical trials or by extrapolation from animal models. The dosage and mode of administration will depend on the individual. Generally, the compositions are administered at a dose between 0.1 mg/kg and 10 mg/kg. Typically, the pharmaceutical composition is administered by injection, either intravenously, subcutaneously or intraperitoneally. It may also be possible to obtain compositions which may be topically or orally administered, or which may be capable of transmission across mucous membranes. If administered by continuous infusion, the infusion may proceed at a dose between 0.05 and 1 mg/kg/hour.

Before administration to patients, formulants and excipients, well known in the art, are preferably added to the pharmaceutical composition. Additionally, pharmaceutical compositions can be chemically modified by covalent conjugation to a polymer to increase there circulating half-life. Polymers, and methods to attach them to peptides, are shown in U.S. Pat. Nos. 4,766,106; 4,179,337; 4,495,285; and 4,609,546, and include polyoxyethylated polyols and PEG.

Monoclonal antibodies against human CD40 are described in U.S. Pat. No. 5,677,165. Antibodies against CD86 can be made by similar methods. The CTLA4-Ig molecule can be made by methods well known in the art. These peptides can be linked to a carrier, for example, keyhole limpet hemocyanin, to increase the immunogenicity and the production of antibodies to the immunogen.

The present invention will now be illustrated by reference to the following examples which set forth particularly advantageous embodiments. However, it should be noted that these embodiments are illustrative and are not to be construed as restricting the invention in any way.

### BRIEF DESCRIPTION OF THE FIGURES

FIG. 1 shows a schematic drawing of the first step in the diabody construction, exchange of the V-regions.

FIG. 2 shows a schematic drawing of the exchange of the linker, the second step in the diabody construction.

FIG. 3 shows a schematic drawing of the strategy applied for cloning of the anti-CD40/anti-CD86 diabody.

FIG. 4 shows a BIAcore sensogram showing the capability of the diabody to bind the CD86-Ig and CD40-Ig antigen simultaneously. From the periplasmic fraction, injected at t=l110 (sec) on a surface containing 6537 Response Units (RU) CD86-Ig, 1300 RU diabody was captured at t=230 (see), as can be seen by the difference in response after the first wash and the normalized signal before injection. At t=360 (sec) CD40-Ig was injected during 120 sec. The diabody, arrested with one binding domain to the CD86-Ig, reacted with the other domain to CD40-Ig yielding 550 RU captured antigen (difference in signal between the second wash at t=480 (sec) and before the second injection at t=360 (sec)).

FIG. 5 shows the results of a T cell activation experiments in which T cells are stimulated with allogeneic monocytes. T cell activation is partially inhibited by blocking CD80 and CD86 with CTLA4-Ig or an antagonistic anti-CD40 monoclonal antibody M3 alone, but almost completely blocked when CTLA4-Ig and antagonistic anti-CD40 monoclonal antibody M3 are combined.

FIG. 6 shows the results of a T cell restimulation experiments in which T cells are stimulated with allogeneic monocytes in the presence of blocking agents during primary stimulation and analysed for proliferative capacity during restimulation in the absence of blocking agents. The presence of CTLA4-Ig alone only results in a slight alloantigen-specific hypo-responsiveness, whereas the combination of CTLA4-Ig with CsA or the antagonistic anti-CD40 monoclonal antibody M3 results in T cell unresponsiveness. T cell responses to the control third-party alloantigen-expressing monocytes is not affected. Closed bars are the T cell responses to the alloantigen used in the primary culture, open bars represent T cell responses to third-party alloantigen-expressing monocytes.

FIG. 7 shows the results of a T cell restimulation experiments in which T cells are stimulated with allogeneic PBMC in the presence of blocking agents during primary stimulation and analysed for proliferative capacity during restimulation in the absence of blocking agents. The presence of antagonistic monoclonal antibody to CD40 and antagonistic monoclonal antibody to CD86, with or without antagonistic monoclonal antibody to CD80 results in alloantigen-specific T cell unresponsiveness. The presence of antagonistic monoclonal antibody to CD40 alone or in combination with antagonistic monoclonal antibody to CD80 only results in partial inactivation of the T cells.

### Examples

Materials and Methods:

p-CD40 and CD86 extracellular domain Ig fusion proteins

In several experiments fusion proteins of lymphocyte cell surface receptors and the Fc-region of human IgG were used. These extracellular domain (ED) fusion proteins (ED-Ig fusion proteins) have been generated by fusion of the nucleic acid sequence encoding the extracellular domain of the cell surface receptors generated by PCR amplification based on published cDNA sequences to the CH1/hinge-CH3 region (Fc) of human IgG1 based on the sequence by Ellison et al. (NAR 10:4071 (1982)). ED-Ig fusion proteins were expressed in Sf9 insect cells and were used as conditioned medium or after purification by affinity chromatography using protein A.

Cell lines and culture conditions

The B-cell line JY was cultured in T75 culture flasks routinely (Costar, Cambridge, Mass., U.S.A.) in Iscove's modified Dulbecco's medium (IMDM) to which 50 μg/ml gentamycin and 10% foetal calf serum was added (FCS) (Hyclone, Logan, Utah U.S.A.). The cells were cultured in a humidified incubator at 37° C. and 5% CO₂. Every week the cells were split (1/20 to 1/100). To store the cell line, ampoules were made containing 5-10×10⁶ cells/ml Hank's balanced salt solution HBSS supplemented with 20% FCS and 10% DMSO and stored in the liquid nitrogen.

Lymphocyte isolation and stimulation

Peripheral blood mononuclear cells (PBMC) were isolated from heparinized blood from healthy donors by Ficoll-Hypaque density centrifugation and re-suspended in complete medium consisting of RPMI 1640 (Gibco, Paisley, UK) supplemented with 2 mM L-Glutamine, streptomycin (100 mg/ml), penicillin (100 U/ml) and 5% heat-inactivated autologous plasma. Enriched monocyte preparations were prepared by rosetting of PBMC with AET-treated sheep red blood cells and removal of E-rosetting cells on Ficoll-Hypaque density gradients, followed by cold aggregation of monocytes as essentially described by Zupo et al. (Eur. J. Immunol. 21:351 (1991)). T cells were further purified from the PBMC preparations by depletion of monocytes, B cells and NK cells using Lympho-Kwik T (One Lambda, Los Angeles, Calif., U.S.A.) according to the manufacturers protocol.

For primary mixed lymphocyte cultures with enriched monocytes and purified T cells, 0.5-1×10⁶/ml purified T cells and 0.1-0.2×10⁶/ml monocytes were cultured in 200 ml complete culture medium in 96-well plates for 6 days in the presence or absence of blocking agents in concentrations ranging from 1-10 μg/ml. For the last 8 hours of the culture period, cells were pulsed with 1 mCi [3H]-thymidine (Amersham International, Amersham, UK). Cells were harvested on glass fiber filters by using a Skatron automatic cell harvester, and radioactivity on the paper was counted in a liquid scintillation counter. For re-stimulation experiments, the T cells (0.5-1×10⁶/ml) and monocytes from the same donor (0.1 to 0.2×10⁶/ml) were cultured for 6 days in 1 ml complete medium in 24-well plates in the presence or absence of blocking agents in concentrations ranging from 1-10 μg/ml and in the presence or absence of cyclosporin A (CsA) at a concentration of 400 ng/ml. After 6 days, the remaining cells were collected and cultured for an additional 2 days, before re-stimulation for 3 days in the absence of blocking agents. T cell proliferation was determined as described above for primary mixed lymphocyte cultures.

Mixed lymphocyte cultures were also performed with non-separated PBMC. In these experiments, fresh PBMC (1×10⁶/ml) as responder cells and PBMC that were pre-activated with human IL-4(20 μg/ml) and human GM-CSF (100 U/ml) (0.1×10⁶/ml) as stimulator cells were cultured for 6 days in 1 ml complete culture medium in 24-well plates in the presence or absence of blocking agents in concentrations ranging from 1-10 μg/ml. After 6 days the remaining cells were collected and cultured for an additional 3 days, before the allo-reactive T cells were re-stimulated with the same PBMC that were pre-activated with human IL-4 (20 μg/ml) and human GM-CSF (100 U/ml) (0.1×10⁶/ml) as stimulator cells for 3 days in the absence of blocking agents. T cell proliferation was assayed by [3H]-thymidine incorporation as described above.

Polymerase chain reaction

To amplify DNA fragments, polymerase chain reactions (PCR) were performed. A typical PCR reaction mix contained: 0-10 mM MgCl₂, 50 mM KCl, 10 mM Tris-HCl pH 9.0, 1.0% Triton X-100, 0.25 mM dNTP each, 25 pmol primer/100 μl reactions mix, 1-1000 ng DNA/100 μl reaction mix and 2.5 U Taq polymerase. Reactions were run using a Perkin Elmer thermocycler (Perkin Elmer Corp, Norwalk Conn.). A stantard PCR scheme consisted of one step for 2-5 min at 95° C. to denature the DNA, followed by 20-40 cycles of 1 min at 95° C., 1 min at 55° C. and 1-4 min at 72° C. After the final step an extension step was performed for 7 min at 72° C.

Flow cytometric analysis (FACS)

Cells (0.1-0.2×10⁶/sample) were incubated for 20 min at 4° C. with the specific monoclonal antibody (0.1-1 mg/sample). After washing with FACS buffer (PBS pH 7.4 1% BSA 0.1% NaN₃), the cells were incubated for another 20 min at 4° C. with goat anti-mouse antibodies conjugated to fluorescein isothiocyanate (FITC) or phycoerythin (PE). The cells were washed with FACS buffer and finally suspended in FACS buffer containing 0.5% paraformaldehyde and analysed with a FACScan flow cytometer (Becton Dickinson). The specific binding of the monoclonal antibodies is expressed as the mean fluorescent intensity in arbitrary units. A similar protocol was used to test the single chain antibody expressing phage particles. In this case detection was done by using an un-conjugated sheep anti-M13 antibody (Pharmacia AB, Uppsala Sweden), followed after washing by incubation with donkey anti-sheep conjugated to FITC (Sigma Chemical Co. St. Louis, Mo., U.S.A.). Likewise a similar protocol was used to demonstrate the biological activity of the diabody and triabody constructs. In these experiments detection was done by incubation of the cells with the diabody or triabody constructs followed by incubation with one of the ED-Ig fusion proteins, followed by incubation with an FITC-conjugated anti-human IgG antiserum.

SDS-PAGE and Western blotting analysis

To analyse the expressed constructs SDS-PAGE and Western blot analysis was performed. Briefly, samples were boiled for 5 min in 0.8% sodium dodecyl sulfate (SDS) and 1 mM dithiothreitol (DTT). Subsequently the samples were run on a 15% SDS-polyacrylamide gel electrophoresis (SDS-PAGE; 2 h 100 V). After electrophoresis the gel was electroblotted to a nitrocellulose filter or stained with 0.1% coommassie blue in 10% methanol and 10% acetic acid. Electroblotting was done in 25 mM Tris-HCl, 192 mM glycine and 10% methanol pH 8.3 for 1 h at 100 V; 4° C. After blotting the nitrocellulose filter was blocked with 1% BSA in PBS-Tween (0.05%) for 1 hour at room temperature. Subsequently the blot was incubated at room temperature in PBS-Tween with a anti c-myc antibody for detection. After incubation with a second antibody (peroxidase labelled), the blots were stained by 4-chloro-naphtol.

BIACore analysis

Analysis were performed on the Pharnacia Biosensor 2000. The CM chip was activated with 0.2 M EDC/0.05 M NHS for 5 min. Subsequently coupling of ligand was done in 10 mM NaAc pH5.0 during 5 min (0.1 mg Ig construct/ml). This was followed by loading of the ScFv, Mab or dia/triabody constructs in various concentrations in PBS buffer. In case of the dia/triabody constructs the chip was washed followed by loading of the second Ig construct. The sensor surface was regenerated with 0.1 M NaOH.

### Example 1

Single chain antibody fragments expressing phage from monoclonal antibodies to human CD40 and CD86 were generated as follows. For the generation of a single chain antibody fragment (ScFv) of the anti-CD40 monoclonal antibody 5D12 both the VH and VL region were amplified by PCR, followed by a second assembly PCR to connect both regions. For this purpose 4 primers were designed (SEQ ID NO: 1-4). SEQ ID NO: 1 contains a HindIII and SfiI restriction site for cloning purposes followed by a degenerated sequence annealing to the 5′ VH region of 5D12. SEQ ID NO: 2 contains a degenerate sequence for the 3′ part of the VH region followed by a sequence encoding a ((Gly)₄Ser)₃ linker and the 5′ part of the VL regions. SEQ ID NO: 3 is a degenerated primer having homology with the 5′ part of the VL region, while the last primer (SEQ ID NO: 4) contains a NotI restriction site and anneals to the 3′ part of the VL region. Briefly, these primers were used to separately PCR amplify the VH and VL regions of monoclonal antibody 5D12. As template for this PCR reaction we used a plasmid containing the VH or VL regions of 5D12 (VH: SEQ ID NO: 5 and VL: SEQ ID NO: 6). The cDNA obtained in this PCR step was gel purified and used in an assembly PCR resulting in the linkage of the V region through the (Gly₄Ser)₃ linker. Subsequently the obtained single chain 5D12 construct was digested with the restriction enzymes HindHIII and NotI, followed by ligation in pGEM-13Zf (Promega Madison U.S.A.). The ligation was transformed in DH5α and plated on LB plates. By sequencing of several clones, a correct 5D12 ScFv clone was found (SEQ ID NO: 7).

For the generation of ScFv's reactive with human CD86, the same primer set as for 5D12 was used. All the steps in the generation of the ScFv of the anti-CD86 monoclonal antibody Fun-1 were performed as described above for the 5D12 ScFv reactive with human CD40. The V regions of Fun-1 (VH: SEQ ID NO: 8; VL: SEQ ID NO: 9) were used as template to obtain the anti-CD86 ScFv construct (SEQ ID NO: 10).

### Example 2

Construction of bi-specific diabody molecules capable of binding to human CD40 and human CD 86:

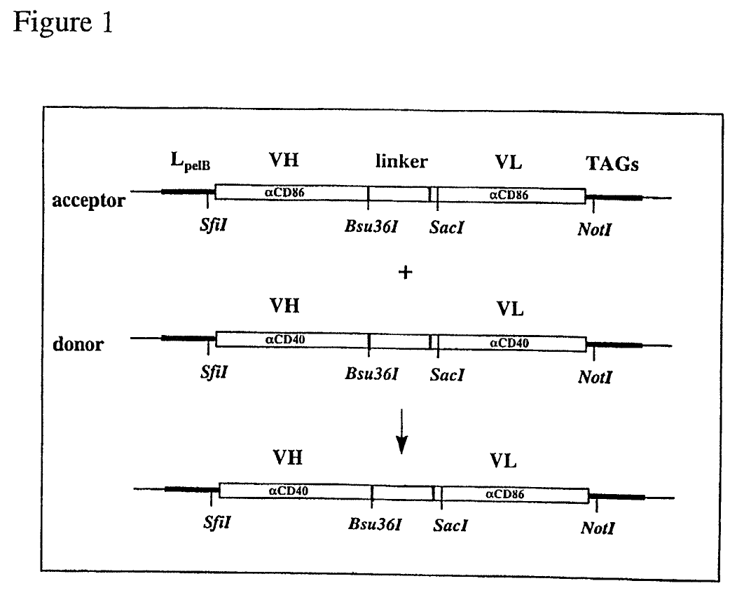

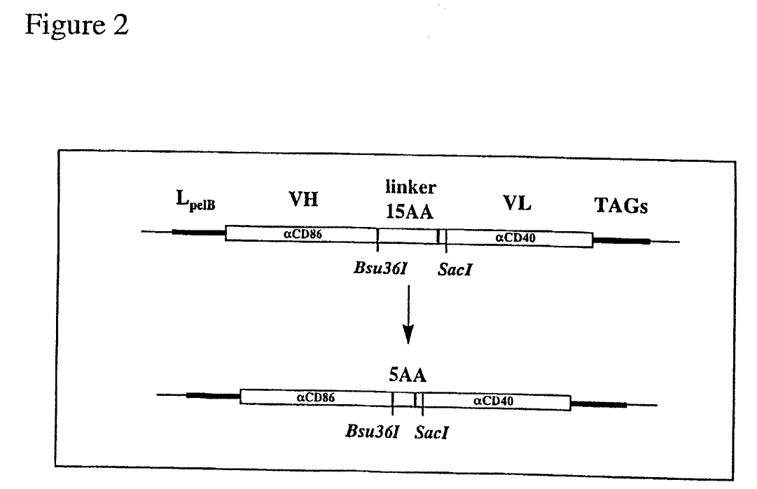

Bi-specific bivalent molecules were generated by shortening the flexible linker sequence in the anti-CD40 ScFv and in the anti-CD86 ScFv, from fifteen residues to five (Gly₄Ser) and by cross-pairing the variable heavy and light chain domains from the two single chain Fv fragments with the different antigen recognition. The construction was performed in three steps. The light chain variable fragments were exchanged in the ScFv constructs from anti-CD86 (aCD86) and anti-CD40 (aCD40) by using restriction enzyme sites located in the 5′ end (SacI at nucleotide numbers 7 to 12 of VL) and just outside the 3′ part of the light chain variable gene (Notl) (see FIG. 1). In the following step the 15-residue linker of the chimeric constructs VH-aCD86/15AA-linker/VL-CD40 (coded 7.2/15/40) and VH-aCD40/15AA-linker/VL-aCD86 (coded 40/15/7.2) was replaced by the 5 residue linker (Gly4Ser) by using sites located in the 3′ part of VH (Bsu361 at nucleotide number 335 of the anti-CD40 VH or at 371 of the anti-CD86 VH to number +2 in the linker sequence) and the 5′ part of VL end (Sacl at nucleotide number 7 to 12 both VL's) (see FIG. 2). Finally, both chimeric cassettes were combined in the vector pUC 119-fabsol (a pUC 119 derivative similar to pUC 119His6mycXba (Low et al., J. Mol. Biol. 260:359 (1996)), but with all Apall-sites in the vector backbone deleted by in vitro mutagenesis) containing a bi-cistronic expression cassette. The subcloning was performed in two steps. First, the aCD86/5 AA-linker/aCD40-construct was cloned in pUC119-fabsol using the restriction sites SfiI and NotI. Subsequently the ScFv cassette of aCD40/5 AA-linker/aCD86 was amplified with the following oligonucleotide primers: 5′-TCT CAC AGT GCA CAG GTG CAG CTG CAG GAG TCT GG-3′ (SEQ ID NO: 11) and 5′-CGT GAG AAC ATA TGG CGC GCC TTA TTA CCG TTT GAT TTC CAG GTT GGT GCC-3′ (SEQ ID NO: 12). These primers contain an ApaLI-and an AscI-site respectively (underlined). The amplified PCR-fragment was digested with ApaLI and AscI, and ligated in the pUC119-fabsol plasmid containing the aCD86/5AA-linker/aCD40-construct. A diabody-producing clone containing both ScFv cassettes was identified and used for expression of the recombinant diabody molecule (pUC119-fabsol-CD40/CD86) (5D12VH+FUNVL: SEQ ID NO: 13; FUNVH+5D12VL: SEQ ID NO: 22).

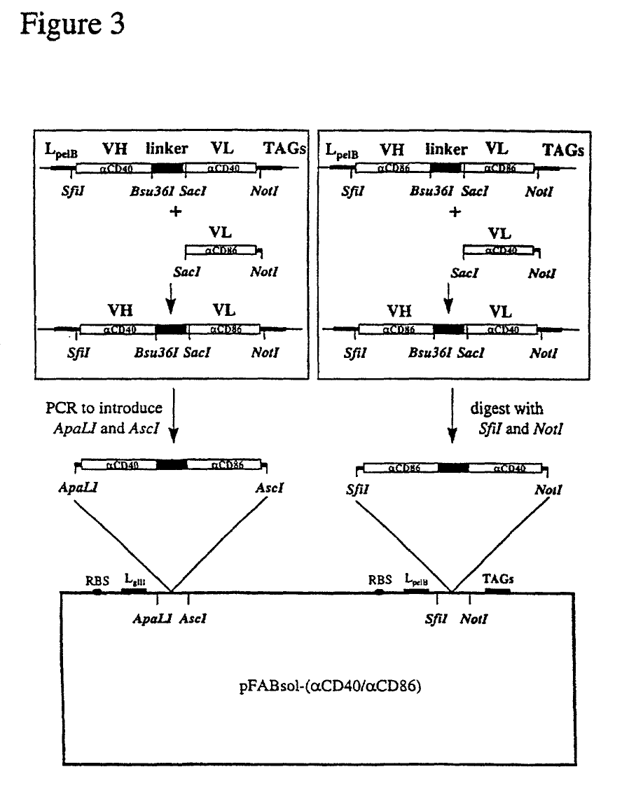

The whole cloning strategy to obtain the bi-specific diabody molecule based on antagonistic anti-CD40 Mabs and antagonistic anti-CD86 Mab Fun-1, capable of binding to human CD40 and human CD86 molecule on antigen presenting cells, is summarized in FIG. 3. The same procedure is also used for the generation of a bi-specific diabody molecule based on antagonistic anti-CD40 Mabs and antagonistic Mabs reactive with both CD80 and CD86, capable of binding to human CD40, human CD80 and human CD86 molecule on antigen presenting cells.

### Example 3

Expression, isolation and characterization of bi-specific diabody molecules capable of binding to human CD40 and human CD86:

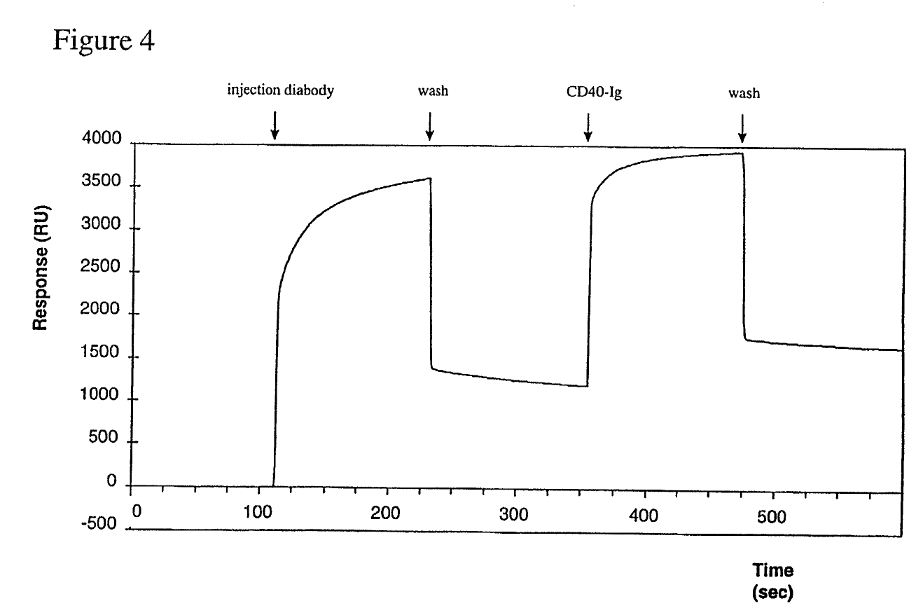

For the production of the bi-specific diabody molecule capable of binding to human CD40 and human CD86, the plasmid containing the aCD86/aCD40-bicistronic expression cassette described in Example 2 above was used. Fifty ml of 2YT medium (100 μg/ml amp, 0.1% glucose) was inoculated (1 v/v %) with a saturated culture (16 hours grown at 30° C.). After 5 hours growth at 30° C. and with a culture having an optical density at 600 nm of 0.9, the diabody production was induced by adding IPTG to a concentration of 1 mM. Cultivation was continued for 4 hours at 20° C. and the cells from 15 ml culture were pelleted (10 minutes at 1100 g at room temperature). Supernatant and cells were stored at −20° C. until further processed. The remaining 35 ml culture was grown at 20° C. for another 16 hours. Cells were pelleted as described above. Periplasmic fractions were prepared by resuspending the cells in 0.53 ml cold TES-buffer (20 mM Tris-HCl; 0.5 mM EDTA; 0.5 M sucrose pH 8.0). The mixture was incubated for 2 minutes on ice, subsequently 0.59 ml cold three fold diluted TES was added and the incubation was prolonged for 30 minutes. The spheroplasts were centrifuged for 15 minutes at 1100 g at 4° C. and the supernatant containing the periplasmic proteins was collected. The pellet fraction was resuspended in 0.75 ml TES/MgSO₄ (TES-buffer; 15 mM MgSO₄) and incubated for 30 minutes on ice. Spheroplasts were pelleted for 15 minutes at 1100 g at 40° C. and the supernatant added to the first supernatant fraction. The total periplasmic fraction was cleared again (15 minutes at 1100 g at 4° C.) and dialyzed against PBS. All fractions were analyzed on PAGE and Western blot with the anti-c-myc antibody for detection. The highest concentration of ScFv was found in the periplasmic fraction prepared from the culture after 4 hours induction, and to some degree in the medium fraction of the culture induced for 20 hours. The functionality of the produced diabody was tested in BIAcore. Purified CD86-Ig was immobilized on the surface of a CM-hip, yielding 6500 RU (Response Units) of coupled protein. Injection of the periplasmic fraction for 120 sec with a flow rate of 10 μl/min resulted in the capture of approx. 1200 RU diabody. Subsequently CD40-Ig was injected under the same conditions as the diabody resulting in the binding of an additional 540 RU antigen. This experiment demonstrated the capability of the produced diabody molecule to bind CD40 and CD86 simultaneously (see FIG. 4).

### Example 4

Construction of bi-specific and tri-specific triabody molecules capable of binding to human CD40 and CD86 or human CD40, CD80and CD86:

The construction of bi-specific and tri-specific triabody molecules is analogous to the scheme described above for the diabody, except that the linker has to be deleted (zero residue linker). This is accomplished by in vitro mutagenesis, using single stranded phagemid DNA and oligonucleotides encoding the mutation. Using ScFv constructs of antagonistic monoclonal antibodies to human CD40, CD80 and CD86 at least two gene constructs are possible: (i) VHαCD40/0/VLαCD80-VHαCD80/0/VLαCD86-VHαCD86/0/VLαCD40 and; (ii) VHαCD40/0/VLαCD86-VHαCD86/0/VLαCD80-VHαCD80/0/VLαCD40. All VH/VL-combinations are made by exchanging VH- and VL-domains in the constructs such as described above for bi-specific diabodies having the 5 amino acid linkers, applying the strategy described above using SfiI and Bsu36I to exchange VH regions, and SacI and NotI to exchange VL regions. Subsequently, the three ScFv-cassettes are cloned in a single expression module encoding a tricistronic mRNA. This DNA will serve as template for an oligo-directed in vitro mutagenesis procedure, to delete the 5 residue linker in one up to three VH-VL-pairs. The various triabodies that are made, may differ in binding characteristics due to other orientations of the ScFv domains and in linker length. Only one of the three ScFv cassettes is provided with the previously mentioned tag sequences. In order to drive triabody formation as well as to maintain stability, disulfide bridges can be introduced by adding cysteine residues at the carboxyterminus or within the V-regions.

### Example 5

Construction of a fusion molecule consisting of an antagonistic anti-CD40 monoclonal antibody linked by its C-terminal residue to the extracellular domain of human CTLA4 capable of binding to CD40, CD80 and CD86 can be carried out as follows. The conceptual therapeutic agent is a fusion protein combining the high affinity and specificity of CTLA4 for both CD80 and CD86 with an antagonistic anti-CD40 monoclonal antibody. This fusion molecule is produced in stable, active form as a complete anti-CD40 monoclonal antibody to which the extracellular domain of human CTLA4 (CTLA4ED) is C-terminal linked by a flexible linker. The construction of the anti-CD40 antibody attached by its Fc part to the extracellular domain of CTLA4 is done by the following PCR and cloning steps. The VH and CH1 regions of anti-CD40 together with a leader sequence are amplified using the oligonucleotides 5′ GCG CGA ATT CAT GGA CAT GAG GGT CCC CGC 3′ (SEQ ID NO: 14) and 5′ AGA TTT GGG CTC AAC TTT CTT GTC CAC 3′ (SEQ ID NO: 15). This is followed by amplification of the CH2 and CH3 regions of human IgG using the oligonucleotides 5′ GTG GAC AAG AAA GTT GAG CCC AAA TCT 3′ (SEQ ID NO: 16) and 5′ GCGC GAA TTC TTA AGC GGC CGC AGA TCC GCC GCC ACC CGA CCC ACC TCC GCC CGA GCC ACC GCC ACC TTT ACC CGG AGA CAG 3′ (SEQ ID NO: 17). After removing of the primers a second PCR is done to assemble both PCR products to obtain a full-length 5D12 heavy chain. The obtained PCR product is gel purified and cloned in pCR Script using the Stratagene cloning kit. Briefly, the PCR product is incubated with plasmid together with T4 ligase and SrfI for 1 h at room temperature, after which the entire sample is transformed in Xl1Blue E. coli cells. The cells are plated on LB plates containing 100 μg ampiciline/ml, 20 μg IPTG/ ml and 20 μg Xgal/ml. After incubation o/n at 37° C. putative white clones are analyzed for having an insert. Clones containing inserts are analyzed by cycle sequencing using M13 and M13 reverse primers. After confirming the correct sequence, the anti-CD40 heavy chain is cloned using the EcoRI restriction site in the bicistronic baculovirus expression plasmid pAcUW51 (Pharmingen) after the p10 promoter. The in this way obtained construct already contains C-terminal a flexible (Gly₄Ser)₃ linker after which the CTLA4ED part was cloned. Therefore the CTLA4ED part is amplified with the oligonucleotides 5′ GCGC GCG GCC GCA ATG CAC GTG GCC CAG CCT G 3′ (SEQ ID NO: 18) and 5′ GCGC GCG GCC GC CTA GTC AGA ATC TGG GCA CGG TTC 3′ (SEQ ID NO: 19) by PCR, gel purified and cloned after the heavy chain of 5D12 using the NotI cloning site. After confirmation by sequence analysis of this step the light chain is cloned. This is done by using a plasmid which already contained the VL region of 5D12 attached to a human CL region. So using the oligonucleotides 5′ GCGC GGATCC ATG GAC ATG AGG GTC CCC GC 3′ (SEQ ID NO: 20) and 5′ GCGC GGATCC CTA ACA CTC TCC CCT GTT GAA GC 3′ (SEQ ID NO: 21) the light chain of 5D12 is amplified and cloned in the constructed pAcUW51 expression plasmid using the BamHI cloning site after the polyhedrin promoter. After DNA sequence analysis a correct clone is obtained. After this confirmation, the expression plasmid containing the 5D12 CTLA4ED construct is introduced into Sf9 insect cells along with the viral AcNPV wild-type DNA using the BaculoGold transfection system of Pharmingen. Recombinant virus is plaque-purified and the integrity of the expression cassette is checked by PCR and cycle sequencing. For protein production, insect cells are used. These cells can grow in suspension in serum-free medium, and are the best known secretors of heterologous proteins. The fusion protein is purified from serum-free conditioned medium by S. aureus protein A affinity chromatography (Harlow and Lane, 1988). Purity is checked by SDS-PAGE and by western blotting under reducing and non-reducing conditions to assess the extent of dimerization.

### Example 6

Effects of blocking the CD4L-CD40 and/or CD80/86-CD28 pathway on the activation of T cells:

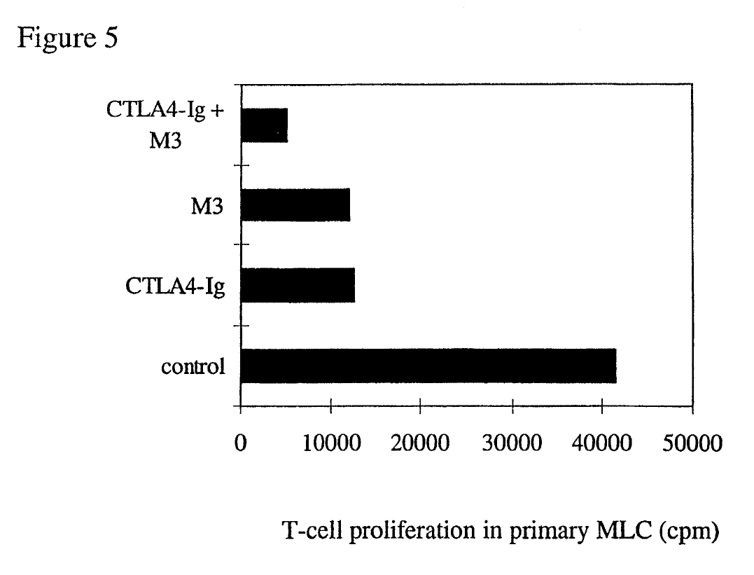

It has been demonstrated extensively that blocking the CD80/86 pathway results in an inhibition of activation of T cells (reviewed in Van Gool et al., Res. in Immunol. 146:183 (1995)). However, under a number circumstances, blocking of the CD80/86 interaction does not result in complete prevention of T cell activation. This is exemplified by FIG. 5, in which purified human T cells are stimulated with allogeneic monocytes as detailed above under materials and methods. Addition of CTLA4-Ig (blocking both CD80 and CD86) only resulted in a partial inhibition of the alloantigen-specific T cells. Surprisingly, addition of the antagonistic anti-CD40 monoclonal antibody M3 also resulted in a partial inhibition of T cell activation to the same extend as CTLA4-Ig. Even more surprisingly, the combination of the antagonistic anti-CD40 monoclonal antibody M3 with CTLA4-Ig resulted in nearly complete blockade of T cell activation.

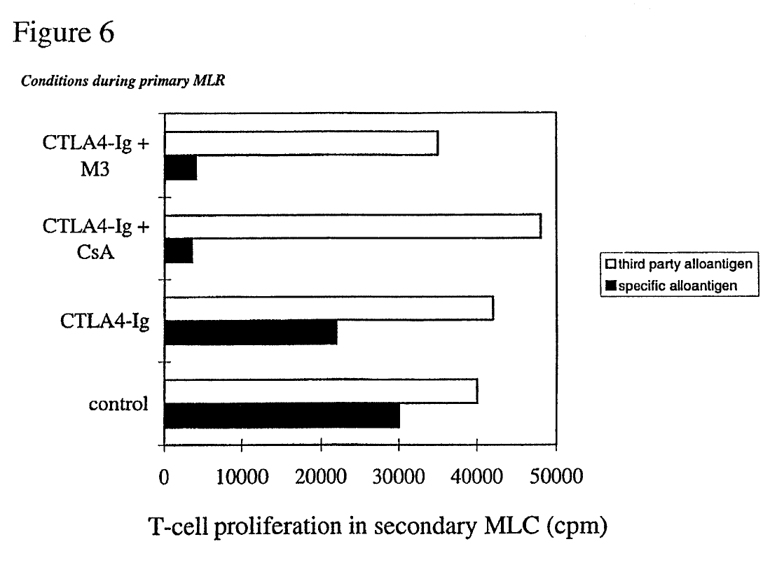

It has also been demonstrated that a blockade of CD80/86 in combination with CsA results in antigen-specific T cell unresponsiveness (reviewed in Van Gool et al., Res. in Immunol. 146:183 (1995)). This has been demonstrated in mixed lymphocyte cultures, in which blocking agents were added during a primary stimulation with the alloantigen, followed by a short rest period and subsequent restimulation with the same alloantigen in the absence of blocking agents. FIG. 6 indeed shows that addition of CTLA4-Ig plus CsA, but not CTLA4-Ig alone to purified human T cells that are stimulated with allogeneic monocytes results in alloantigen-specific T cell unresponsiveness (solid bars). The response to unrelated third party alloantigen-expressing monocytes in unchanged (open bars). FIG. 6 also shows that the addition of CTLA4-Ig plus anti-CD40 monoclonal antibody M3 alone to purified human T cells that are stimulated with allogeneic monocytes also results in alloantigen-specific T cell unresponsiveness (solid bars). Again, this unresponsiveness to the alloantigen of the first culture is specific, since the response to unrelated third party alloantigen-expressing monocytes in unchanged (open bars).

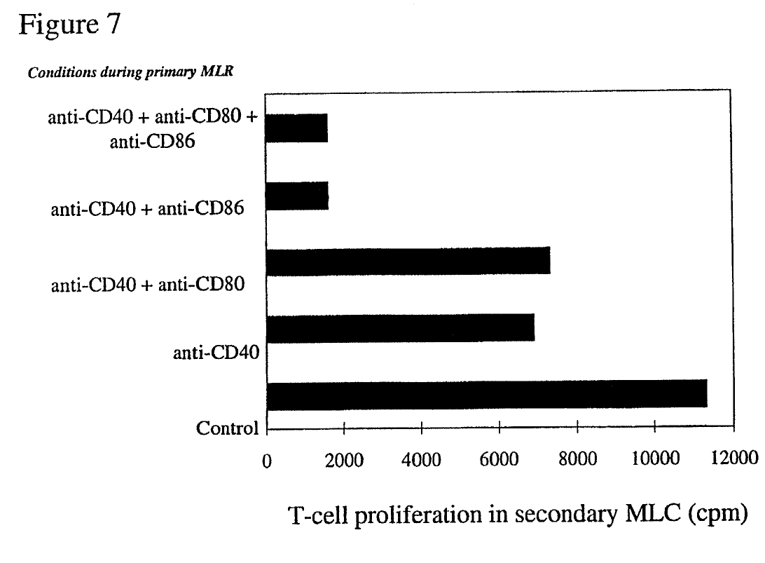

In another set of experiments (FIG. 7) it is shown that a combination of blocking CD40 with the antagonistic anti-CD40 monoclonal antibody 5D12 and blocking of CD80 and CD86 with antagonistic monoclonal antibodies, results in alloantigen-specific T cell unresponsiveness when tested in MLC experiments using PBMCs as detailed above in the materials and methods section. Surprisingly, alloantigen-specific T cell unresponsiveness was also induced when the anti-CD40 monoclonal antibody was combined with the antagonistic anti-CD86 monoclonal antibody without blocking the CD80 costimulatory receptor. In contrast, the combination of the anti-CD40 monoclonal antibody and the anti-CD80 monoclonal antibody, without blocking the CD86 receptor resulted only in T cell hypo-responsiveness to the same level as with the antagonistic anti-CD40 monoclonal antibody alone. This was surprising, since it has extensively been demonstrated that blocking of both CD80 and CD86 always results in more complete inhibition that with either alone. This demonstrates that blocking the CD86-CD28 costimulatory interaction together with blocking the CD40L-CD40 costimulatory interaction with one therapeutic molecule such as described in the above examples has a strong potential for immunotherapy of T cell mediated diseases.

### Example 7

Gene Therapy to Produce the Proteins of the Invention

Another embodiment of the invention includes gene constructs that direct the expression in vivo of the diabodies of the invention which bind to the human CD40 and CD86 antigens (or the diabodies or triabodies which bind to human CD40, CD80 and CD86, or the fusion protein including anti-CD40 and CTLA4-Ig) located on the surface of antigen presenting cells. The gene constructs can be introduced by well-known methods using viral vectors, including a retrovirus, an adenovirus, a parvovirus or any other vector permitting cellular transfer of the gene constructs, or by incorporation of the gene construct into liposomes with or without the viral vector. The gene constructs can also be transfected into cells ex vivo, using known methods including electroporation, calcium phosphate transfection, micro-injection, or incorporation of the gene constructs into liposomes followed by transfection. The cells are then introduced into the patient for antibody expression in vivo.

The gene constructs are made by the cloning strategy as set forth above for construction of the diabodies and triabodies of the invention. The heavy and light chain genes can be placed in one plasmid construct either under separate promoter control or under one promoter in a dicistronic arrangement. The antibody gene fragments can also be placed under control of promoters that allow the turning on and off of the gene expression with appropriate exogeneous factors such as steroids or metal ions.

These gene constructs can be prepared as plasmids for direct DNA delivery into host cells or tissues. With additional manipulations using techniques known in the field of genetic therapy, the gene constructs can also be coupled with a suitable viral particle, including a retrovirus, an adenovirus, or a parvovirus which allow gene delivery through viral infection. Any of these gene constructs can also be used to transfect cells suitable for antibody expression ex vivo. Following transfection, the cells are introduced into the subject where the antibody is expressed.

To test whether these gene constructs can direct the expression of the desired antibody or antibody fragments, suitable gene constructs or viral particles are first used to transfect or infect appropriate host cells. Culture supernatants of the transfected/infected cells are collected an appropriate period post transfection/transfection and tested for antibody expression in ELISA to detect the presence of the antibody and its ability to bind CD40 and CD86, or CD40, CD80 and CD86, if applicable. Further testing can include the measurement of the antibody affinity and the ability to compete with the parent antibodies for binding to the antigens.

To test whether the gene constructs or the cells transfected/infected as above can direct the antibody expression in vivo, plasmid gene constructs (or the transfected/infected cells) can be administered to BALB/c mice intramuscularly, either formulated with phosphate buffered saline or with suitable liposome preparation, or in the case of viral vectors, using proper infection protocols. The treated animals are analyzed for expression of the diabodies, triabodies or fusion proteins, as applicable, either with tissue section staining, or by expression thereof in blood.

1. A soluble molecule capable of binding to the human CD40 antigen and to the human CD86 antigen, said antigens being located on the surface of human lymphocytes.

2. A soluble binding molecule according to claim 1, which is an antibody containing an antigen-binding site of an antibody to CD40 and an antigen-binding site of an antibody to CD86.

3. An antibody molecule according to claim 2, which is a trispecific diabody capable of binding to CD40 and to both CD80 and CD86, in particular by containing the antigen-binding site of an antibody to CD40 and the antigen-binding site of an antibody which is cross-reactive with CD80 and CD86.

4. An antibody molecule according to claim 2, which is a bispecific diabody capable of binding to human CD40 and to human CD86, in particular by containing the antigen-binding site of an antibody to CD40 and the antigen-binding site of an antibody to CD86.

5. An antibody molecule according to claim 2, which is a trispecific triabody capable of binding to CD40, CD80 and CD86, in particular by containing the antigen-binding site of an antibody to CD40, the antigen-binding site of an antibody to CD80 and the antigen-binding site of an antibody to CD86.

6. A soluble binding molecule according to claim 1 or 3, which is capable of binding to CD86 by means of the extracellular domain of human CTLA-4.

7. An antibody according to claim 4 or 5, wherein the antibody to CD86 is the antibody Fun-1.

8. An antibody according to any one of claims 2 to 5 wherein the antibody to CD40 is an antagonistic antibody to CD40.

9. An antibody according to any one of claims 2-5, wherein the antibody to CD40 is a non-stimulatory antagonistic antibody to CD40.

10. A recombinant vector comprising the nucleotide sequences encoding the binding molecule fragments according to any one of claims 1-5 operably linked to regulating sequences capable of expressing the antibody molecule in a host cell.

11. A host cell stably transformed with the vector according to claim 10.

12. A method of producing a recombinant molecule capable of binding to the human CD40 antigen and to at least the human CD86 antigen, comprising culturing a host cell and isolating the binding molecule from the culture medium.

13. A pharmaceutical composition for induction of T cell tolerance, containing a therapeutically effective amount of the binding molecule according to any one of claims 1-5 and a pharmaceutically acceptable carrier.

14. A method for treating T cell mediated immune responses, the method comprising administering to a patient in need of such treatment a therapeutically effective amount of a pharmaceutical composition according to claim 13.

15. A method for preventing allograft transplant rejection, the method comprising administering to a patient in need of such treatment a therapeutically effective amount of a pharmaceutical composition according to claim 13.

16. A method for preventing xenotransplant rejection, the method comprising administering to a patient in need of such treatment a therapeutically effective amount of a pharmaceutical composition according to claim 13.

17. A method for the induction of T cell tolerance, the method comprising administering to a patient in need of such treatment a therapeutically effective amount of a pharmaceutical composition according to claim 13.

18. A method for the induction of allo-transplant or xeno-transplant tolerance, the method comprising administering to a patient in need of such treatment a therapeutically effective amount of a pharmaceutical composition according to claim 13.

19. A method for preventing or treatment of autoimmune diseases such as rheumatoid arthritis, multiple sclerosis and psoriasis, the method comprising administering to a patient in need of such treatment a therapeutically effective amount of a pharmaceutical composition according to claim 13.

20. A method for treating T cell mediated immune responses to gene therapy vectors or vehicles, the method comprising administering to a patient in need of such treatment a therapeutically effective amount of a pharmaceutical composition according to claim 13.

21. A method for treating T cell mediated immune responses to therapeutic molecules, the method comprising administering to a patient in need of such treatment a therapeutically effective amount of a pharmaceutical composition according to claim 13.

22. Gene constructs encoding ligands capable of binding to CD40 and CD86, or to CD40, CD80 and CD86.

23. The gene constructs of claim 22 wherein the ligands encode triabodies or diabodies.

24. The constructs of claim 22 wherein the ligand capable of binding to CD86 is a CTLA4-Ig fusion protein.

25. The gene constructs of any of claims 22 to 24 wherein the gene constructs are incorporated in a plasmid or a viral vector.

26. A method of transfecting cells with the gene constructs of any of claims 22 to 24.

27. Cells transfected or infected with the gene constructs of any of claims 22 to 24.

28. The method of claim 25 wherein the transfection or infection is done ex vivo or in vivo.

29. The method of claim 27 wherein the transfection is done ex vivo by electroporation, calcium phosphate transfection, micro-injection or by incorporating the gene constructs into suitable liposomes.

30. The method of claim 27 wherein the infection is done in vivo or ex vivo by incorporating the gene constructs into a retrovirus, adenovirus or a parvovirus vector, or by incorporating the gene constructs, or the gene constructs with a viral vector, into a suitable liposome.


SEQUENCE LISTING (22 sequences)

SEQ ID NO: 1 - DNA, 52 bp, human
gcgcaggcttggcccagccggccatggcccaggtsmarctgcagsagtctgg

SEQ ID NO: 2 - DNA, 89 bp, human
gtgagctcgatgtccgatccgccaccgccagagccacctccgcctgaaccgcctccacctgaggagacggtgaccgtggtcccttggcc

SEQ ID NO: 3 - DNA, 24 bp, Artificial Sequence
  human
gacatcgagctcacycagtctcca

SEQ ID NO: 4 - DNA, 42 bp, human
gcgcgcggccgcccgtttkatttccagsttggtgcctccacc

SEQ ID NO: 5 - DNA, 339 bp, human
caggtgcaactcgtggagtctggacctggcctggtgaaaccctcacagagcctgtccatcacatgcactgtctctgggttctcattatccagatatagtgtatactgggttcgccagcctccaggaaagggtctggagtggctgggaatgatgtggggtggtggatccacagactataattcagctctcaaatccagactgaccatcagcaaggacacctcgaagaaccaggtcttcttaaaaatgaacagtctgcgagctgaggacacagccatgtactactgtgtcagaaccgatggggactactggggtcaaggaaccaccgtcaccgtctcctca

SEQ ID NO: 6 - DNA, 339 bp, human
gacctccagctgacccagtctccactctccctgcctgtcagtcttggagatcgagcctccatctcttgcagatctagtcagagccttgtaaacagtaatggaaacacctatttacattggtacctgcagaagccaggccagtctccaaagctcctgatctacaaagtttccaaccgattttctggggtcccagacaggttcagtggcagtggatcagggacagatttcacactcaagattagcagagtggaggctgaggatgtgggagtttattactgctctcaaagtacacatgttccgtggacgttcggtggaggcaccaagctggaaatcaaacga

SEQ ID NO: 7 - DNA, 723 bp, human
caggtgcagctgcaggagtctggacctggcctggtgaaaccctcacagagcctgtccatcacatgcactgtctctgggttctcattatccagatatagtgtatactgggttcgccagcctccaggaaagggtctggagtggctgggaatgatgtggggtggtggatccacagactataattcagctctcaaatccagactgaccatcagcaaggacacctcgaagaaccaggtcttcttaaaaatgaacagtctgcgagctgaggacacagccatgtactactgtgtcagaaccgatggggactactggggccaagggaccacggtcaccgtctcctcaggtggaggcggttcaggcggaggtggctctggcggtggcggatcggacatcgagctcactcagtctccactctccctgcctgtcagtcttggagatcgagcctccatctcttgcagatctagtcagagccttgtaaacagtaatggaaacacctatttacattggtacctgcagaagccaggccagtctccaaagctcctgatctacaaagtttccaaccgattttctggggtcccagacaggttcagtggcagtggatcagggacagatttcacactcaagattagcagagtggaggctgaggatgtgggagtttattactgctctcaaagtacacatgttccgtggacgttcggtggaggcaccaagctggaaataaaacgg

SEQ ID NO: 8 - DNA, 375 bp, human
gaggtccaactgcagcagtctggacctgagctggagaagcctggcgcttcagtgaagatatcctgcaaggcttctggttactcattcactgactacaacatgaactgggtgaagcagagcaatggaaagagccttgagtggattggaaatattgatccttactatggtggtactagttacaatcagaagttcaagggcaaggccacattgactgtagacaaatcctccagcacagcctacatgcagctcaacagcctgacatctgaagactctgcagtctatttctgtgcaagatgggactataggtacgacgacgggagggcttactatgttatggacttctggggtcaaggaacctcagtcaccgtctcctca

SEQ ID NO: 9 - DNA, 339 bp, human
gagctccagatgacccagtctccatcatctctggctgcgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacacattttactctgaccgtcagcagtgtgcaagctgaagacctggcagtttattactgtcatcaatacctctactcgtggacgttcggtggaggcaccaacctggaaatcaaacgg

SEQ ID NO: 10 - DNA, 759 bp, human
caggtccaactgcagcagtctggacctgagctggagaagcctggcgcttcagtgaagatatcctgcaaggcttctggttactcattcactgactacaacatgaactgggtgaagcagagcaatggaaagagccttgagtggattggaaatattgatccttactatggtggtactagttacaatcagaagttcaagggcaaggccacattgactgtagacaaatcctccagcacagcctacatgcagctcaacagcctgacatctgaagactctgcagtctatttctgtgcaagatgggactataggtacgacgacgggagggcttactatgttatggacttctggggccaagggaccacggtcaccgtctcctcaggtggaggcggttcaggcggaggtggctctggcggtggcggatcggacatcgagctcacccagtctccatcatctctggctgcgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacacattttactctgaccgtcagcagtgtgcaagctgaagacctggcagtttattactgtcatcaatacctctactcgtggacgttcggtggaggcaccaacctggaaatcaaacgg

SEQ ID NO: 11 - DNA, 35 bp, human
tctcacagtgcacaggtgcagctgcaggagtctgg

SEQ ID NO: 12 - DNA, 51 bp, human
cgtgagaacatatggcgcgccttattaccgtttgatttccaggttggtgcc

SEQ ID NO: 13 - DNA, 764 bp, human
gtgaaaaaattattattcgcaattcctttagttgttcctttctattctcacagtgcacaggtgcagctgcaggagtctggacctggcctggtgaaaccctcacagagcctgtccatcacatgcactgtctctgggttctcattatccagatatagtgtatactgggttcgccagcctccaggaaagggtctggagtggctgggaatgatgtggggtggtggatccacagactataattcagctctcaaatccagactgaccatcagcaaggacacctcgaagaaccaggtcttcttaaaaatgaacagtctgcgagctgaggacacagccatgtactactgtgtcagaaccgatggggactactggggccaagggaccacggtcaccgtctcctcaggtggaggcggttcagacattgagctcacccagtctccatcatctctggctgcgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacacattttactctgaccgtcagcagtgtgcaagctgaagacctggcagtttattactgtcatcaatacctctactcgtggacgttcggtggaggcaccaacctggaaatcaaacggtaataaggcgcgcc

SEQ ID NO: 14 - DNA, 30 bp, human
gcgcgaattcatggacatgagggtccccgc

SEQ ID NO: 15 - DNA, 27 bp, human
agatttgggctcaactttcttgtccac

SEQ ID NO: 16 - DNA, 27 bp, human
gtggacaagaaagttgagcccaaatct

SEQ ID NO: 17 - DNA, 82 bp, human
gcgcgaattcttaagcggccgcagatccgccgccacccgacccacctccgcccgagccaccgccacctttacccggagacag

SEQ ID NO: 18 - DNA, 31 bp, human
gcgcgcggccgcaatgcacgtggcccagctt

SEQ ID NO: 19 - DNA, 36 bp, human
gcgcgcggccgcctagtcagaatctgggcacggttc

SEQ ID NO: 20 - DNA, 31 bp, human
gcggcggatccatggacatgagggtccccgc

SEQ ID NO: 21 - DNA, 33 bp, human
gcgcggatccctaacactctcccctgttgaagc

SEQ ID NO: 22 - DNA, 879 bp, human
atgaaatacctattgcctacggcagccgctggattgttattactcgcggcccagccggccatggcccaggtccaactgcagcagtctggacctgagctggagaagcctggcgcttcagtgaagatatcctgcaaggcttctggttactcattcactgactacaacatgaactgggtgaagcagagcaatggaaagagccttgagtggattggaaatattgatccttactatggtggtactagttacaatcagaagttcaagggcaaggccacattgactgtagacaaatcctccagcacagcctacatgcagctcaacagcctgacatctgaagactctgcagtctatttctgtgcaagatgggactataggtacgacgacgggagggcttactatgttatggacttctggggccaagggaccacggtcaccgtctcctcaggcggtggcggatcggacattgagctcactcagtctccactctccctgcctgtcagtcttggagatcgagcctccatctcttgcagatctagtcagagccttgtaaacagtaatggaaacacctatttacattggtacctgcagaagccaggccagtctccaaagctcctgatctacaaagtttccaaccgattttctggggtcccagacaggttcagtggcagtggatcagggacagatttcacactcaagattagcagagtggaggctgaggatgtgggagtttattactgctctcaaagtacacatgttccgtggacgttcggtggaggcaccaagctggaaataaaacgggcggccgcagaacaaaaactcatctcagaagaggatctgaatggggccgcacatcaccatcatcaccattaataagaatccbct